(19) United States
(12) Patent Application Publication
(10) Pub. No.: US 2026/0225974 A1
(43) Pub. Date: Aug. 6, 2026

(54) PROCESS FOR CONTROLLING HEAT TRANSFER AND GENERATING POWER

(71) Applicant: UOP LLC, Rosemont, IL (US)

(72) Inventors: Srinivasan Ramanujam, Bengaluru (IN); Ian G. Horn, Streamwood, IL (US); Rohit Mudadi, Gurgaon (IN); Nageswara Rao Chitta, Gurgaon (IN); James W. Harris, Palatine, IL (US); Phillip F. Daly, Palatine, IL (US)

(21) Appl. No.: 19/425,091

(22) Filed: Dec. 18, 2025

Related U.S. Application Data
(60) Provisional application No. 63/739,415, filed on Dec. 27, 2024.

(51) Int. Cl. C07C 5/10; F01K 7/16; F22B 1/16; F22G 1/00

(52) U.S. Cl. CPC C07C 5/10; F01K 7/16; F22B 1/167; F22G 1/00

(57) ABSTRACT

A process for controlling heat transfer in a hydrogenation process is provided. A hydrocarbon feed stream is hydrogenated in a hydrogenation reactor in the presence of hydrogen and a hydrogenation catalyst to produce a hydrogenated effluent. In an embodiment, the hydrogen is green hydrogen. A first feed stream is taken from the hydrogenated effluent and said first feed stream is sent to a steam generator to produce a steam stream. A pressure of the steam stream produced in the steam generator is adjusted to control the temperature of the first feed stream and the first feed stream is then charged to a second reactor.

### FIELD

The field relates to the process for heat recovery in a hydrogenation process.The field may particularly relate to the process for controlling heat transfer and generating power in a hydrogenation process.

### BACKGROUND

Hydrogen is expected to have significant growth potential because it is a clean burning fuel. Aggressive carbon emission reduction targets and rising carbon penalties to meet the Paris Climate Agreement are anticipated to drive a hydrogen-based economy in the near future.

Hydrogen production processes based on steam reforming, autothermal reforming, partial oxidation, or gasification of hydrocarbon or carbonaceous feedstocks are significant emitters of CO₂. Government regulations and societal pressures are increasingly taxing or penalizing CO₂ emissions or incentivizing CO₂ capture. Consequently, there is a significant interest in lowering the cost of hydrogen production using these processes and recovering the byproduct CO₂ for subsequent geological sequestration. Green hydrogen generated by renewable energy sources such as solar, wind, and water, which does not involve the production of CO₂, could meet projected global energy demand in the future and play a vital role in reducing carbon emissions. The recently renewed interest in alternative energy sources and energy carriers opens up new prospects for this process to be applied as a feed system for fuel cells, power generation and many more applications.

A huge regional disparity exists in the cost of production of hydrogen. A number of technologies have been developed for transporting hydrogen, including NH₃, liquid H₂, and liquid organic hydrogen carrier (LOHC) to address this disparity. Toluenemethylcyclohexane (MCH) is expected to be a significant player in the LOHC space considering numerous advantages, such as easy integration with existing fuel sector supply chain and distribution network, utilization in idle refinery assets, flexibility for coprocessing, and higher relative handling safety.

LOHC involves the reversible dehydrogenation reaction of methylcyclohexane (MCH) to produce toluene and hydrogen through the methylcyclohexane dehydrogenation process. It has been proposed as a solution for the storage, transportation, and distribution of hydrogen produced from renewable energy sources. For power generation, the hydrogen from this process is usually compressed for the downstream power generation unit. Usually, the purity requirement for a power generation unit is very stringent. Due to the relatively high cost associated with green hydrogen production, it is necessary to recover almost all hydrogen.

While offtakers prefer stable supply of hydrogen to the toluene hydrogenation unit, the unit has hourly and seasonal fluctuations of the renewable power generation. It requires the toluene hydrogenation unit to ramp up/down to synchronize the MCH production with the fluctuation in the upstream hydrogen source. Customers require flexibility in ramping up and ramping down the capacity of the process unit, almost from 20% to 100% of unit capacity daily, due to the cyclical nature of the green hydrogen production. Competing LOHC technologies in the market are net power consumers and do not use specialty heat exchangers to improve the heat integration of the process.

Conventional controls around steam generators result in design and safety concerns for specialty exchangers that require multiple relief valves on reactors for a blocked-out scenario. In a blocked-out scenario, the flow of a hydrogenation reactor effluent is blocked during the splitting of the effluent stream into a bypass stream and a stream which is fed to a steam generator and a series control valve is provided to control the flow of the hydrogenation reactor effluent towards the steam generator. Alternatively, removing these specialty exchangers from the loop would streamline the flow design and allow the use of conventional heat exchangers capable of handling blocked-out conditions. However, this simplification would reduce overall heat recovery, leading to lower steam and power output, and consequently affecting the carbon intensity of the hydrogenation process.

Accordingly, it would be desirable to have a process which eliminates safety concerns in having specialty exchangers for the efficient heat recovery while also handling the daily feed variation in a hydrogenation process.

### SUMMARY

A process for controlling heat transfer in a hydrogenation process is provided. A first hydrocarbon feed stream is hydrogenated in a hydrogenation reactor in the presence of hydrogen and a hydrogenation catalyst to produce a hydrogenated effluent stream. In an embodiment, the hydrogen is green hydrogen. The hydrogenated effluent stream is cooled in a steam generator to produce a cooled hydrogenated effluent stream and a steam stream.. A pressure of the steam stream produced in the steam generator is adjusted to control the temperature of the cooled hydrogenated effluent stream.

In an embodiment, the hydrogenated effluent is split into a bypass stream and a first exchanger inlet stream, and the first exchanger inlet stream is cooled in the steam generator. The bypass stream is combined with the cooled hydrogenated effluent stream downstream of the steam generator to produce a combined feed stream and the combined feed stream is charged to the second hydrogenation reactor. The temperature of the combined feed stream is also controlled by adjusting the flow rate of the bypass stream around the steam generator. A pressure of the steam stream produced in the steam generator is adjusted to control the temperature of the first feed stream, thereby controlling the heat transfer in the steam generator. More preferably, the pressure of the steam stream is increased to control the heat transfer in the steam generator. The pressure of the steam stream may be increased in small increments. By adjusting the pressure of the steam stream produced in the steam generator, a temperature differential is reduced in order to reduce the heat transfer in the steam generator.

The hydrocarbon feed preferably comprises toluene which is hydrogenated to methylcyclohexane (MCH) in a liquid organic hydrogen carrier (LOHC) process. MCH may be further transferred to a second location to dehydrogenate the MCH to toluene. In an exemplary embodiment, a hydrogenation section comprises more than one hydrogenation reactor, preferably four hydrogenation reactors.

In an embodiment, the hydrogenated effluent throughput in the bypass stream is varied by maintaining the size of a bypass valve such that a maximum of 40% of the hydrogenated effluent throughput can go through the bypass stream instead of in conventional schemes where, 100% of the hydrogenated effluent throughput could go through the bypass stream. The hydrogenated effluent throughput in the bypass stream is varied to maintain a minimum pressure drop through one or more tubes of a heat exchanger even at a high turndown of up to 25%. This is achieved by adjusting the opening of a bypass valve.

The proposed solution helps to eliminate safety and design concerns in having specialty exchangers for the efficient heat recovery while also handling the daily feed variation. The process further involves minimizing the adjustment in pressure of the steam generator to about 345 kPa (50 psi). Minimizing the steam pressure change ensures reliable operation of the steam generator.

Another aspect of the present disclosure involves a process for generating power in a hydrogenation process. The process comprises monitoring a dew point margin of a hydrocarbon feed stream at an inlet of a hydrogenation reactor and maintaining a dew point margin by adjusting a reactor inlet temperature of the hydrogenation reactor. The hydrocarbon feed stream is hydrogenated in the hydrogenation reactor in the presence of hydrogen and a hydrogenation catalyst to produce a hydrogenated effluent and the hydrogenated effluent is then passed through a superheater exchanger to produce a superheated steam stream. The superheated steam stream is further used to generate power in a turbine. The reactor inlet temperature of the hydrogenation reactor is adjusted to maintain the dew point margin of the hydrocarbon feed stream based on a dew point margin set point above the reactor inlet temperature. The dew point margin set point is about 11°C (20°F) to about 14°C (25°F).

The dew point monitoring at the inlet of the hydrogenation reactor allows monitoring of the dew point margin sufficiently to ensure maximum capture and superheating of the steam stream to recover power. By actively monitoring the dew point margin of the hydrocarbon feed stream at the inlet of the hydrogenation reactor, there is less risk of condensation in the hydrogenation reactor which may lead to hot spots or catalyst damage in the reactors when recovering the heat produced in the superheater exchanger.

### DEFINITIONS

As used herein, “liquid organic hydrogen carrier” or “LOHC” refers to a hydrogenated organic substrate selected from monocyclic, polycyclic, heterocyclic and homocyclic organic compounds that can be processed to release chemically bound hydrogen via dehydrogenation, and which are liquid at standard temperature and pressure.

As used herein, the term “wt %” as used here is equivalent to “percent by weight”.

### BRIEF DESCRIPTION OF THE DRAWINGS

FIG. 1 is an illustration of one embodiment of a process for controlling heat transfer in a hydrogenation process according to the present disclosure.

FIG. 2 is an illustration of one embodiment of a process for generating power in a hydrogenation process according to the present disclosure.

FIG. 3 shows a dynamic simulation of the control system according to the present disclosure.

### DESCRIPTION

The present disclosure involves a process for controlling heat transfer in a hydrogenation process. A first hydrocarbon feed stream is hydrogenated in a hydrogenation reactor in the presence of hydrogen and a hydrogenation catalyst to produce a hydrogenated effluent stream. In an embodiment, the hydrogen is green hydrogen. The hydrogenated effluent stream is cooled in a steam generator to produce a cooled hydrogenated effluent stream and a steam stream. A pressure of the steam stream produced in the steam generator is adjusted to control the temperature of the cooled hydrogenated effluent stream.

The hydrogenated effluent is split into a bypass stream and a first exchanger inlet stream, and the first exchanger inlet stream is cooled in the steam generator. The bypass stream is combined with the cooled hydrogenated effluent stream downstream of the steam generator to produce a combined feed stream and the combined feed stream is charged to the second hydrogenation reactor. The temperature of the combined feed stream is also controlled by adjusting the flow rate of the bypass stream around the steam generator. In an embodiment, the reactor inlet temperature is controlled by split range of a first feed stream bypass around the steam generator and the steam pressure controller setpoint on the outlet of the steam generator.

A pressure of the steam stream produced in the steam generator is adjusted to control the temperature of the first feed stream, thereby controlling the heat transfer in the steam generator. By adjusting the pressure of the steam stream produced in the steam generator, a temperature differential is reduced in order to reduce the heat transfer in the steam generator. The proposed solution helps to eliminate safety and design concerns in having specialty exchangers for the efficient heat recovery while also handling the daily feed variation.

In an embodiment, the pressure of the steam stream is increased to control the heat transfer in the steam generator. By increasing the pressure of the steam stream produced in the steam generator, the degree of heat transfer to the first feed stream decreases.

Another unique feature of the disclosure is that the hydrogenated effluent throughput in the bypass stream can be varied by maintaining the size of a bypass valve such that a maximum of only about 40% of the hydrogenated effluent throughput can go through the bypass stream instead of as in conventional schemes wherein 100% of the hydrogenated effluent throughput goes through the bypass stream. The hydrogenated effluent throughput in the bypass stream is varied to maintain a minimum pressure drop through one or more tubes of a heat exchanger even at a high turndown of up to 25%. This is achieved by adjusting the opening of a bypass valve. The process further involves minimizing the adjustment in pressure of the steam generator to about 345 kPa (50 psi). Minimizing the steam pressure change ensures reliable operation of the steam generator.

Another aspect of the present disclosure involves a process for generating power in a hydrogenation process. The process comprises monitoring a dew point margin of a hydrocarbon feed stream at an inlet of a hydrogenation reactor and maintaining a dew point margin by adjusting a reactor inlet temperature of the hydrogenation reactor. The hydrocarbon feed stream is hydrogenated in the hydrogenation reactor in the presence of hydrogen and a hydrogenation catalyst to produce a hydrogenated effluent and the hydrogenated effluent is then passed through a superheater exchanger to produce a superheated steam stream. The superheated steam stream is further used to generate power in a turbine. The reactor inlet temperature of the hydrogenation reactor is adjusted to maintain the dew point margin of the hydrocarbon feed stream based on a dew point margin set point above the reactor inlet temperature. The dew point margin set point is about 11°C (20°F) to about 14°C (25°F).

The hydrocarbon feed preferably comprises toluene which is hydrogenated to methylcyclohexane (MCH) in a liquid organic hydrogen carrier (LOHC) process. MCH may be further transferred to a second location to dehydrogenate the MCH to toluene. It can be performed with minimal to no by-products, thereby ensuring minimal loss of hydrogen (green or blue hydrogen). MCH acts as a liquid organic hydrogen carrier, and it can be transferred in storage vessels and/or pipelines for several thousands of miles to the final destination with very minimal to no degradation. The LOHC process helps address the supply and demand gap of blue and green hydrogen as well as the huge differential cost of production between regions.

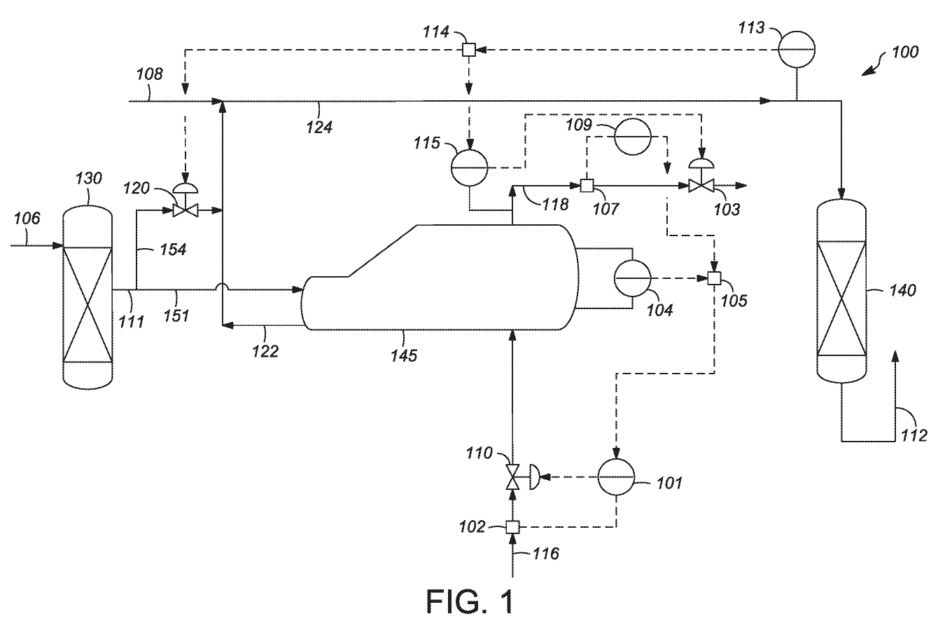

FIG. 1 illustrates one embodiment of a process for controlling heat transfer in a hydrogenation process 100. In an aspect, the hydrogenation process 100 represents the hydrogenation cycle of the LOHC loop. The hydrogenation process 100 comprises two hydrogenation reactor(s). A hydrocarbon feed stream comprising a dehydrogenated hydrocarbon is passed to a first hydrogenation reactor 130. The hydrocarbon feed stream may comprise one or more hydrogen carriers for carrying the hydrogen after hydrogenation. The hydrocarbon feed stream may comprise a dehydrogenated hydrocarbon such as toluene. The dehydrogenated hydrocarbon carries the hydrogen after hydrogenation. The hydrocarbon feed stream may comprise heavier dehydrogenated C12 to C16 unsaturated hydrocarbons such as C12 to C16 aromatics, preferably unsaturated C14 hydrocarbons. C12 to C16 unsaturated hydrocarbons may include multi-ring aromatics like methyl-substituted biphenyls and methyl-substituted fluorene. C14 unsaturated hydrocarbons may include C14 aromatics such as dimethyl-biphenyls and methyl-fluorenes. The heavier dehydrogenated C12 to C16 hydrocarbons may be used as a hydrogen carrier along with the other dehydrogenated hydrocarbons such as the toluene.

The hydrocarbon feed stream may be taken from an external source such as a storage tank (not shown). In an exemplary embodiment, the hydrogenation process 100 comprises two hydrogenation reactors, a first hydrogenation reactor 130 and a second hydrogenation reactor 140. Fewer or more hydrogenation reactors than two may be utilized.

The hydrocarbon feed stream is split into a first hydrocarbon feed stream and a second hydrocarbon feed stream. The first hydrocarbon feed stream is combined with hydrogen to form a combined feed stream in line 106. In an embodiment, the hydrogen is green hydrogen. The combined feed stream in line 106 can optionally be further heated in a combined feed exchanger (not shown) by a last effluent stream (not shown) obtained from a last hydrogenation reactor to obtain the desired initial temperature for the hydrogenation reaction The combined feed exchanger may be a shell and tube type heat exchanger or a welded plate type heat exchanger or a spiral tube heat exchanger. In an embodiment, the combined feed exchanger is a welded plate type heat exchanger.

The combined feed stream in line 106 is sent to the first hydrogenation reactor 130 where toluene is converted to methylcyclohexane in the presence of a first hydrogenation catalyst to provide a first hydrogenated effluent stream in line 111. The first hydrogenated effluent stream in line 111, comprising the converted methylcyclohexane, exits the first hydrogenation reactor 130.

The first hydrogenated effluent stream in line 111 is split into a first bypass stream in line 154 and a first exchanger inlet stream in line 151. In an embodiment, a hydrogenated effluent throughput in the first bypass line 154 varies, such that, at most about 40% of the hydrogenated effluent stream is sent to the first bypass line 154. The hydrogenated effluent throughput in the first bypass line 154 is varied to maintain a minimum pressure drop through one or more tubes of a heat exchanger even at a high turndown of up to about 25%. This is achieved by adjusting the opening of a first bypass valve 120. A first cooled stream in line 122 obtained from the first steam generator 145 is mixed with the bypass stream in line 154 and a second fresh feed stream in line 108 to form a second combined feed stream in line 124. In an embodiment, the second fresh feed stream in line 108 comprises toluene.

The conversion in the first hydrogenation reactor 130 releases a large amount of heat, which must be removed. The first exchanger inlet stream in line 151 is sent to a first steam generator 145 to be cooled with a boiler feed water in line 116 to produce a first steam stream in line 118. In an embodiment, a flow controller 101 is provided to maintain a flow control of the boiler feed water in line 116 to the first steam generator 145 by modulating a boiler feed water valve 110. The boiler feed water line 116 also includes a first flow measuring device 102 which sends a signal to the flow controller 101 to modulate the flow of the boiler feed water to the first steam generator 145.

The pressure of the first steam stream in line 118 produced in the first steam generator 145 is adjusted to control the temperature of the first cooled stream in line 122 which in turn controls the temperature of the second combined feed stream in line 124, thereby controlling the heat transfer in the first steam generator 145. By increasing the pressure of the first steam stream in line 118 produced in the first steam generator 145, the temperature differential between the first exchanger inlet stream in line 151 and the first steam stream in line 118 is reduced in order to reduce the heat transfer in the first steam generator 145. The first steam generator 145 includes a level indicating controller 104 which measures the level of the boiler feed water in the first steam generator 145 and provides an output to a first calculation block 105. In an embodiment, the level indicating controller 104 comprises a measuring device which measures the level of the boiler feed water in the first steam generator 145.

The first calculation block 105 sends the output provided by the level indicating controller 104 in the form of a signal to the flow controller 101 which in turn biases or changes a setpoint for the flow controller 101. The flow controller 101 then measures the flow of the boiler feed water in line 116 using the first flow measuring device 102 and compares this flow against the set point provided by the first calculation block 105. The flow of the boiler feed water in line 116, which is fed to the first steam generator 145, is balanced based on the comparison provided by the flow controller 101.

In an embodiment, the pressure of the first steam stream in line 118 is increased thereby, increasing the temperature of the first steam stream in line 118. The pressure of the first steam stream in line 118 is increased in small increments. By increasing the pressure of the steam stream in line 118 produced in the first steam generator 145, the degree of heat transfer from the first exchanger inlet stream in line 151 decreases. The first steam stream in line 118 includes a second flow measuring device 107 which measures the flow rate of the first steam stream in line 118 produced in the first steam generator 145 and sends the flow rate signal to a flow indicator 109. The flow indicator 109 sends the flow rate from the second flow measuring device 107 to the first calculation block 105.

The first calculation block 105 is a calculation algorithm which takes two inputs and provides a single output. The two inputs fed to the first calculation block 105 include the flow rate signal from the flow indicator 109 which provides the flow rate of the first steam stream in line 118 and the output provided by the level indicating controller 104, which measures the level of the boiler feed water in the first steam generator 145. Based on the two inputs provided to the first calculation block 105, the first calculation block 105 sends a signal to the flow controller 101, which in turn biases a setpoint for the flow controller 101. The flow rate of the first steam stream generated in line 118 is balanced by adjusting the flow rate of the boiler feed water in line 116 to the first steam generator 145. The flow rate measured by the second flow measuring device 107 should be equal to the flow rate measured by the first flow measuring device 102 in order to balance the flow rate of boiler feed water being sent to the first steam generator 145. This is achieved by biasing a set point value for the flow controller 101 which measures the flow rate of the boiler feed water in line 116 and compares this flow rate against the set point provided by the first calculation block 105.

The second flow measuring device 107 and the flow indicator 109 may be provided in a single assembly or a single device. In an embodiment, the second flow measuring device 107 is not provided and the flow indicator 109 directly measures the flow rate of the first steam stream in line 118.

In an embodiment, the adjustment in pressure of the first steam generator 145 is minimized to about 345 kPa (50 psi) in order to ensure reliable operation of the first steam generator 145. The first steam generator 145 and the first bypass control valve 120 are designed such that the variation in steam generation pressure is operated in the pressure range of about 2100 kPa(g) (305 psig) to about 2450 kPa(g) (355 psig) to avoid potential operation issues in the first steam generator 145.

A temperature of the second combined feed stream in line 124 is also controlled by adjusting the flow rate of the first bypass stream in line 154 around the first steam generator 145 using the first bypass control valve 120. The temperature of the second combined feed stream in line 124 is measured by a temperature indicating controller 113. The temperature indicating controller 113 measures the temperature of the second combined feed stream in line 124 and sends a signal to a second calculation block 114.

The second calculation block 114 may be a split range controller which splits the signal provided by the temperature indicating controller 113 between the first bypass control valve 120 and a pressure indicating controller 115. After receiving the signal from the second calculation block 114, the first bypass control valve 120 adjusts the flow rate of the first bypass stream in line 154 which is then mixed with the first cooled stream in line 122. In an embodiment, the pressure indicating controller 115, which also receives a signal from the second calculation block 114, is a pressure indicator controller. The temperature measured by the temperature indicating controller113 is then compared to a setpoint value and, in case the temperature measured by the temperature indicating controller113 is higher than the setpoint value, the pressure indicating controller 115 signals to close a valve 103 on the first steam stream in line 118 in order to increase the pressure in the first steam stream in line 118. According to the setpoint value, the valve 103 on the first steam stream in line 118 is adjusted, i.e. the valve 103 is either opened or closed to control the flow of steam into or out of the process. This action regulates the pressure of the first steam stream in line 118 and helps maintain the desired heat transfer in the first steam generator 145.

The second combined feed stream in line 124 is charged to the second hydrogenation reactor 140 where toluene is converted to additional methylcyclohexane in the presence of a second hydrogenation catalyst to provide a second hydrogenated effluent stream in line 112. The second hydrogenated effluent stream in line 112 exits the second hydrogenation reactor 140 and is further passed to a second steam generator (not shown). In an embodiment, a portion of second hydrogenated effluent stream in line 112 is passed to a superheater exchanger (not shown) to produce a superheated steam stream. The portion of the second hydrogenated effluent stream in line 112 is heat exchanged with the first steam stream in line 118 to produce a superheated steam stream. The superheated steam stream is sent to a turbine to generate power. In an embodiment, the power is generated in a condensing type steam driven turbine.

In an embodiment, the second hydrogenated effluent stream in line 112 may be further processed in additional hydrogenation reactors and further cooled in additional steam generators. The second hydrogenated effluent stream in line 112 may go to the combined feed exchanger (not shown) to recover additional heat to provide a cooled second hydrogenated effluent stream. The cooled second hydrogenated effluent stream may be further cooled and separated in a condenser (not shown), and a separator (not shown) respectively. The separator (not shown) produces a vapor stream and a liquid stream.

In a further embodiment, the vapor stream from the separator (not shown) comprises excess hydrogen from the second hydrogenated effluent stream in line 112 and may be recycled to the first combined feed stream in line 106 via a compressor (not shown). In an additional embodiment, the liquid stream from the separator may be routed to a fractionator (not shown) to remove one or more contaminants from the hydrogenated product stream and to produce a fractionated bottoms liquid stream and a fractioned overhead vapor stream. The fractionated bottoms liquid stream from the fractionator is the purified hydrogenated product stream and is cooled and sent to storage. The purified hydrogenated product stream is transported via ship, barge, rail car, truck, and/or pipeline to another location.

In an embodiment, the purified hydrogenated product stream is sent to a dehydrogenation unit in another location (not shown), where the hydrogenated product stream is dehydrogenated to produce hydrogen and a dehydrogenation unit product stream. In another embodiment, the dehydrogenation unit product stream is transported back to the hydrogenation process 100. In a further embodiment, the hydrocarbon feed stream of the hydrogenation process 100 comprises the dehydrogenation unit product stream.

The first and second hydrogenation catalysts may be any suitable hydrogenation catalysts. The first and second hydrogenation catalysts can be the same or different. The hydrogenation catalyst should have high selectivity, and a low rate of coke lay down. Suitable hydrogenation catalysts may include, but are not limited to, a metal of Group VIII of the Periodic Table and optionally a metal of Group I of the Periodic Table. Suitable hydrogenation catalysts may also include, but are not limited to, 0.05 wt% to 30 wt% of a metal of Group VIII of the Periodic Table and optionally 0.1 wt% to 3 wt% of a metal of Group I of the Periodic Table.

Typical operating pressures for the hydrogenation reactor(s) 130, 140 are in the range of about 1034 kPa(g) (150 psig) to about 6895 kPa(g) (1000 psig), or about 2758 kPa(g) (400 psig) to about 4482 kPa(g) (650 psig). Typical inlet temperatures for all of the reactors are in the range of about 204 °C (400 °F) to about 232 °C (450 °F). Typical outlet temperatures for the hydrogenation reactor(s) 130, 140 are in the range of about 316 °C (600 °F) to about 371 °C (700 °F).

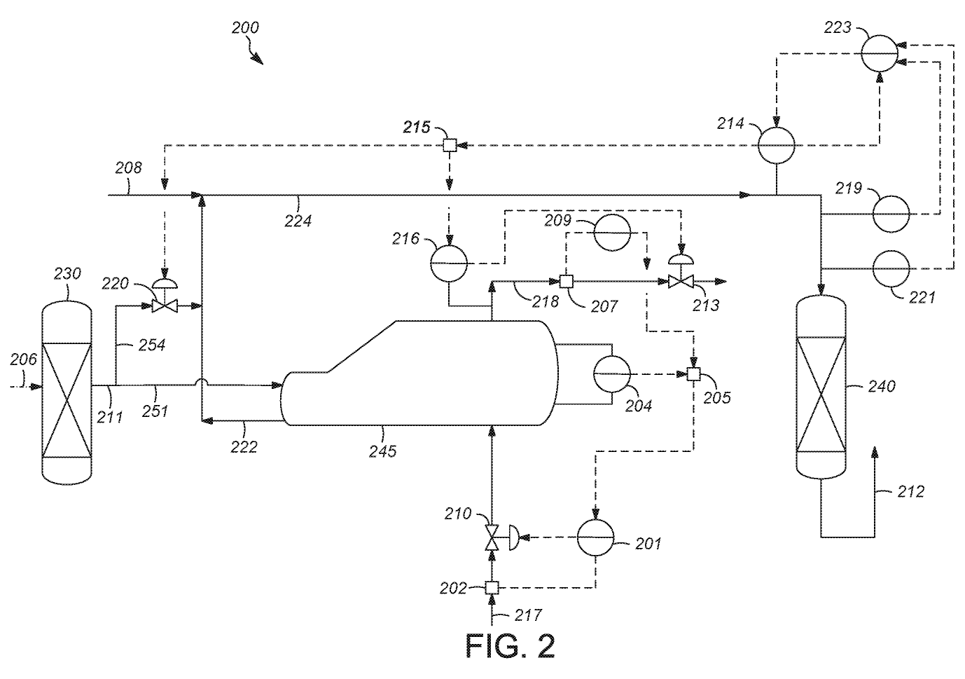

FIG. 2 illustrates one embodiment of a process for generating power in a hydrogenation process 200. In an aspect, the hydrogenation process 200 represents the hydrogenation cycle of the LOHC loop. The hydrogenation process 200 comprises two hydrogenation reactor(s). A hydrocarbon feed stream comprising a dehydrogenated hydrocarbon is passed to a first hydrogenation reactor 230. The hydrocarbon feed stream may comprise one or more hydrogen carriers for carrying the hydrogen after hydrogenation. The hydrocarbon feed stream may comprise a dehydrogenated hydrocarbon such as toluene. The hydrogenated hydrocarbon carries the hydrogen after hydrogenation.

The hydrocarbon feed stream may be taken from an external source such as a storage tank (not shown). In an exemplary embodiment, the hydrogenation process 200 comprises two hydrogenation reactors, a first hydrogenation reactor 230 and a second hydrogenation reactor 240. Fewer or more hydrogenation reactors than two may be utilized.

The hydrocarbon feed stream is split into a first hydrocarbon feed stream and a second hydrocarbon feed stream. The first hydrocarbon feed stream is combined with hydrogen to form a combined feed stream in line 206. In an embodiment, the hydrogen is green hydrogen. The combined feed stream in line 206 can optionally be further heated in a combined feed exchanger (not shown) by a second effluent stream in line 212 to obtain the desired initial temperature for the hydrogenation reaction. The combined feed exchanger may be a shell and tube type heat exchanger or a welded plate type heat exchanger or a spiral tube heat exchanger. In an embodiment, the combined feed exchanger is a welded plate type heat exchanger.

The combined feed stream in line 206 is sent to a first hydrogenation reactor 230 where toluene is converted to methylcyclohexane in the presence of a first hydrogenated catalyst to produce a first effluent stream in line 211. The first effluent stream in line 211, comprising the converted methylcyclohexane, exits the first hydrogenation reactor 230.

The first effluent stream in line 211 is split into a first bypass stream in line 254 and a first exchanger inlet stream in line 251. In an embodiment, a hydrogenated effluent throughput in the first bypass line 254 varies, such that, at most about 40% of the hydrogenated effluent is sent to the first bypass line 254. The hydrogenated effluent throughput in the first bypass stream in line 254 is varied to maintain a minimum pressure drop through one or more tubes of a heat exchanger even at a high turndown of up to about 25%. This is achieved by adjusting the opening of a first bypass valve 220. A first cooled stream in line 222 obtained from the first steam generator 245 is mixed with the bypass stream in line 254 and a second fresh feed stream in line 208 to form a second combined feed stream in line 224. In an embodiment, the second fresh feed stream in line 208 comprises toluene.

The conversion in the first hydrogenation reactor 230 releases a large amount of heat, which must be removed. The first exchanger inlet stream in line 251 is sent to a first steam generator 245 to be cooled with a boiler feed water in line 217 to produce a first steam stream in line 218. In an embodiment, a flow controller 201 is provided to maintain a flow control of the boiler feed water in line 217 to the first steam generator 245 by modulating a boiler feed water valve 210. The line 217 also includes a first flow measuring device 202 which sends a signal to the flow controller 201 to modulate the flow of the boiler feed water to the first steam generator 245.

The pressure of the first steam stream in line 218 produced in the first steam generator 245 is adjusted to control the temperature of the first cooled stream in line 222 which in turn controls the temperature of the second combined feed stream in line 224, thereby controlling the heat transfer in the first steam generator 245. By increasing the pressure of the first steam stream in line 218 produced in the first steam generator 245, the temperature differential between the first exchanger inlet stream in line 251 and the first steam stream in line 218 is reduced in order to reduce the heat transfer in the first steam generator 245. The first steam generator 245 includes a level indicating controller 204 which measures the level of the boiler feed water in the first steam generator 245 and provides an output to a first calculation block 205.

The first calculation block 205 sends the output provided by the level indicating controller204 in the form of a signal to the flow controller 201 which in turn biases or changes a setpoint for the flow controller 201. The flow controller 201 then measures the flow rate of the boiler feed water in line 217 using the first flow measuring device 202 and compares this flow rate against the set point provided by the first calculation block 205. The flow rate of the boiler feed water in line 217, which is fed to the first steam generator 245, is balanced based on the comparison provided by the flow controller 201.

In an embodiment, the pressure of the first steam stream in line 218 is increased thereby, increasing the temperature of the first steam stream in line 218. The pressure of the first steam stream in line 218 is increased in small increments. By increasing the pressure of the steam stream in line 218 produced in the first steam generator 245, the degree of heat transfer from the first exchanger inlet stream in line 251 decreases. The line 218 includes a second flow measuring device 207 which measures the flow rate of the first steam stream in line 218 produced in the first steam generator 245 and sends the flow rate signal to a flow indicator 209. The flow indicator 209 sends the flow rate from the second flow measuring device 207 to the first calculation block 205.

The first calculation block 205 is a calculation algorithm which takes two inputs and provides a single output. The two inputs fed to the first calculation block 205 include the flow rate signal from the flow indicator 209 which provides the flow rate of the first steam stream in line 218 and the output provided by the level indicating controller204 which measures the level of the boiler feed water in the first steam generator 245. Based on the two inputs provided to the first calculation block 205, the first calculation block 205 sends a signal to the flow controller 201 which in turn biases a setpoint for the flow controller 201. The first steam stream generated in line 218 is balanced by adjusting the flow rate of the boiler feed water in line 217 to the first steam generator 245. The flow rate measured by the second flow measuring device 207 should be equal to the flow rate measured by the first flow measuring device 202 in order to balance the flow rate of boiler feed water being sent to the first steam generator 245. This is achieved by biasing a set point value for the flow controller 201 which measures the flow rate of the boiler feed water in line 217 and compares this flow rate against the set point provided by the first calculation block 205.

The second flow measuring device 207 and the flow indicator 209 may be provided in a single assembly or a single device. In an embodiment, the second flow measuring device 207 is not provided and the first steam stream in line 218 is directly sent to the flow indicator 209 to measure the flow of the first steam stream in line 218.

In an embodiment, the adjustment in pressure of the first steam generator 245 is minimized to about 345 kPa (50 psi) in order to ensure reliable operation of the first steam generator 245. The first steam generator 245 and the first bypass control valve 220 are designed such that the variation in steam generation pressure is operated in the pressure range of about 2100 kPa(g) (305 psig) to about 2450 kPa(g) (355 psig)) to avoid potential operation issues in the first steam generator 245.

A temperature of the second combined feed stream in line 224 is also controlled by adjusting the flow rate of the first bypass stream in line 254 around the first steam generator 245 using the first bypass control valve 220. The temperature of the second combined feed stream in line 224 is measured by a temperature indicating controller 214. The temperature indicating controller 214 measures the temperature of the second combined feed stream in line 224 and sends a signal to a second calculation block 215.

The second calculation block 215 may be a split range controller which splits the signal provided by the temperature indicating controller 214 between the first bypass control valve 220 and a pressure indicating controller 216. After receiving the signal from the second calculation block 215, the first bypass control valve 220 adjusts the flow rate of the first bypass stream in line 254 which is then mixed with the first cooled stream in line 222. In an embodiment, the pressure indicating controller 216, which also receives a signal from the second calculation block 215, is a pressure-indicating controller. The temperature measured by the temperature indicating controller214 is then compared to a setpoint value and in case, the temperature measured by the temperature indicating controller214 is higher than the setpoint value, the pressure indicating controller 216 signals to close a valve 213 placed on the first steam stream in line 218 in order to increase the pressure in the first steam stream in line 218. According to the setpoint value, the valve 213 on the first steam stream in line 218 is adjusted i.e., the valve 213 is either opened or closed to control the flow of steam into or out of the process. This action regulates the pressure of the first steam stream in line 218 and helps maintain the desired heat transfer in the first steam generator 245.

The second combined feed stream in line 224 is charged to the second hydrogenation reactor 240, where toluene is converted to additional methylcyclohexane in the presence of a second hydrogenated catalyst to provide a second effluent stream in line 212. The second effluent stream in line 212 exits the second hydrogenation reactor 240.

In an embodiment, a dew point margin of the second combined feed stream in line 224 in the second hydrogenation reactor 240 is monitored at an inlet of the second hydrogenation reactor 240. The dew point margin of the second combined feed stream in line 224 is monitored and maintained by adjusting a reactor inlet temperature of the second hydrogenation reactor 240. The reactor inlet temperature of the second hydrogenation reactor 240 is adjusted to maintain the dew point margin of the second combined feed stream in line 224 based on a dew point margin set point above the reactor inlet temperature. In an embodiment, the dew point margin set point is about 11°C (20°F) to about 14°C (25°F). In an embodiment, the dew point margin of the second combined feed stream 224 is monitored using one or more dew point monitors which are provided at the inlet of the second hydrogenation reactor 240. In an embodiment, the inlet of the second hydrogenation reactor 240 comprises a control system calculation block 223 to determine the dew point condition of the second combined feed stream in line 224 based on temperature, pressure, and composition of the second combined feed stream. The dew point condition of the second combined feed stream in line 224 is measured by using at least three sensors. In an embodiment, the three sensors comprise a composition monitor 219, a pressure monitor 221 and the temperature indicating controller 214.

The control system calculation block 223 makes the calculation of the current conditions such as the temperature, pressure, and composition of the second combined feed stream in line 224 and adds margin to provide a dew point margin temperature setpoint to the temperature indicating controller 214. The signal generated by the control system calculation block 223 is used as the setpoint to the temperature indicating controller 214. The temperature indicating controller 214 adjusts the temperature of the second combined feed stream in line 224 by closing the bypass valve 220 and in split range adjusts the setpoint of the pressure indicating controller 216 to maintain the dew point margin of the second combined feed stream in line 224 entering the second hydrogenation reactor 240.

The composition monitor219 may be an analyzer which analyzes the composition of the second combined feed stream 224 which is fed to the second hydrogenation reactor 240. The pressure monitor 221 provided at the inlet of the second hydrogenation reactor 240 measures the pressure of the second combined feed stream in line 224 before it enters the second hydrogenation reactor 240. The composition monitor219 and the pressure monitor 221 measure the composition and pressure of the second combined feed stream 224 respectively and send a measurement to the control system calculation block 223. In an embodiment, the control system calculation block 223 is a dew point margin which provides an override to an existing set point value of the temperature indicating controller 214. The control system calculation block 223 compares the temperature measurement input from temperature indicating controller214 to a setpoint value and provides a remote setpoint to the temperature indicating controller 214 which maintains the dew point margin and adjusts the temperature of second combined feed stream in line 224 using the second calculation block 215 to control the pressure setpoint for the pressure indicating controller 216 or opening of the first bypass valve 220 from reactor 230.

After monitoring the dew point margin of the second combined feed stream in line 224, the stream undergoes hydrogenation in the second hydrogenation reactor 240 where the toluene is converted to methylcyclohexane in the presence of a first hydrogenated catalyst. The second effluent stream 212, comprising the converted methylcyclohexane, exits the second hydrogenation reactor 240.

A portion of second effluent stream in line 212 is passed to a superheater exchanger (not shown) to produce a superheated steam stream. The superheated steam stream is sent to a turbine to generate power. In an embodiment, the power is generated in a condensing type steam driven turbine. The dew point margin of the second combined feed stream in line 224 is monitored to ensure maximum capture and superheating of the superheated steam stream in order to generate power in the turbine. By actively monitoring the dew point margin of the second combined feed stream in line 224 at the inlet of the second hydrogenation reactor 240, there is less risk of condensation in the second hydrogenation reactor 240 which may lead to hot spots or catalyst damage in the reactors when recovering the heat produced in the superheater exchanger.

The second effluent stream in line 212 exits the second hydrogenation reactor 240 and is then sent to the combined feed exchanger where heat is exchanged with the combined feed stream in line 206. The combined feed exchanger may be a shell and tube type heat exchanger or a welded plate type heat exchanger or a spiral tube heat exchanger. In an embodiment, the combined feed exchanger is a welded plate-type heat exchanger.

The first and second hydrogenation catalysts may be any suitable hydrogenation catalysts. The first and second hydrogenation catalysts can be the same or different. The hydrogenation catalyst should have high selectivity, and a low rate of coke lay down. Suitable hydrogenation catalysts may include, but are not limited to, a metal of Group VIII of the Periodic Table and optionally a metal of Group I of the Periodic Table. Suitable hydrogenation catalysts may also include, but are not limited to, 0.05 wt% to 30 wt% of a metal of Group VIII of the Periodic Table and optionally 0.1 wt% to 3 wt% of a metal of Group I of the Periodic Table.

Typical operating pressures for the hydrogenation reactor(s) 230, 240 are in the range of about 1034 kPa(g) (150 psig) to about 6895 kPa(g) (1000 psig), or about 2758 kPa(g) (400 psig) to about 4482 kPa(g) (650 psig). Typical inlet temperatures for all of the reactors are in the range of about 204 °C (400 °F) to about 232 °C (450 °F). Typical outlet temperatures for the hydrogenation reactor(s) 230, 240 are in the range of about 316 °C (600 °F) to about 371 °C (700 °F).

### EXAMPLES

### EXAMPLE 1

For 86 KMTA H2 of production in a LOHC Toluene Hydrogenation Unit, expected power consumption of the unit is around 13 MW. With the introduction of welded plate or a spiral tube heat exchanger, the overall steam and power production increased by ~30%. Hence, the overall power production is 16.5 MW for 86 KMTA capacity. Overall, the net consumption of the power to the unit will be zero and net export of approximately 3.5 MW of power can be achieved. Thus, there is a negative carbon intensity for this operation. The power generated pays off for itself in approximately a year.

### EXAMPLE 2

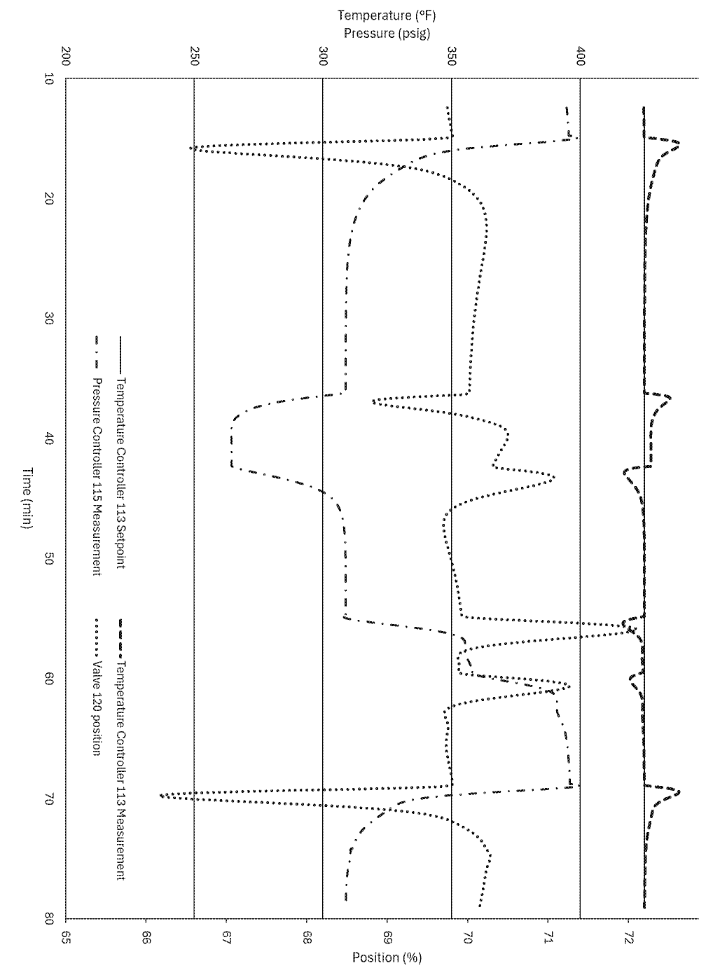

A dynamic simulation of the control system was performed using UniSim Design dynamic simulation mode. This test was designed to confirm the control response of an exemplary embodiment of the process to step changes in unit capacity. The plot of FIG. 3 is temperature, pressure or valve position relative to fully open with step changes in the feed flow rate over time. Changes in feed flow rate require a change in duty of the first steam generator 145. The temperature indicating controller 113 responds to the load change in less than a minute and restores the temperature in a controlled manner over the succeeding four minutes. The pressure indicating controller 115 and first bypass valve 120 work together to maintain control in a smooth and safe manner.

### SPECIFIC EMBODIMENTS

While the following is described in conjunction with specific embodiments, it will be understood that this description is intended to illustrate and not limit the scope of the preceding description and the appended claims.

A first embodiment of the present disclosure a process for controlling heat transfer in a hydrogenation process, the steps comprising, hydrogenating a first hydrocarbon feed stream in a hydrogenation reactor in the presence of hydrogen and a hydrogenation catalyst to produce a hydrogenated effluent stream; cooling the hydrogenated effluent stream in a steam generator to produce a cooled hydrogenated effluent stream and a steam stream; and adjusting a pressure of the steam stream produced in the steam generator to control the temperature of the cooled hydrogenated effluent stream.

An embodiment of the present disclosure is one, any or all of prior embodiments in this paragraph up through the first embodiment in this paragraph, splitting the hydrogenated effluent stream into a bypass stream and a first exchanger inlet stream and cooling the first exchanger inlet stream in the steam generator; and combining the bypass stream with the cooled hydrogenated effluent stream downstream of the steam generator into a combined feed stream and charging the combined feed stream to the second hydrogenation reactor. . An embodiment of the present disclosure is one, any or all of prior embodiments in this paragraph up through the first embodiment in this paragraph wherein the temperature of the combined feed stream is also controlled by adjusting the flow rate of the bypass stream around the steam generator. An embodiment of the present disclosure is one, any or all of prior embodiments in this paragraph up through the first embodiment in this paragraph, further comprising minimizing the adjustment in pressure of the steam generator to about 50 psi. An embodiment of the present disclosure is one, any or all of prior embodiments in this paragraph up through the first embodiment in this paragraph, further comprising varying a hydrogenated effluent throughput in the bypass stream, wherein at most 40% of the hydrogenated effluent is sent to the bypass stream. An embodiment of the present disclosure is one, any or all of prior embodiments in this paragraph up through the first embodiment in this paragraph, wherein the hydrogen is obtained from renewable energy sources. An embodiment of the present disclosure is one, any or all of prior embodiments in this paragraph up through the first embodiment in this paragraph, further comprising monitoring a dew point of the hydrocarbon feed stream at an inlet of the hydrogenation reactor. An embodiment of the present disclosure is one, any or all of prior embodiments in this paragraph up through the first embodiment in this paragraph, further comprising maintaining a dew point margin by adjusting a reactor inlet temperature of the hydrogenation reactor. An embodiment of the present disclosure is one, any or all of prior embodiments in this paragraph up through the first embodiment in this paragraph, further comprising, superheating the steam stream in a heat exchanger with a second hydrogenated effluent stream to produce a superheated steam stream and a cooled second hydrogenated effluent stream; and generating power in a turbine through the superheated steam stream. An embodiment of the present disclosure is one, any or all of prior embodiments in this paragraph up through the first embodiment in this paragraph, wherein the heat exchanger is a superheater exchanger.

A second embodiment of the present disclosure is a process for controlling heat transfer in a hydrogenation process, the steps comprising, hydrogenating a first hydrocarbon feed stream in a hydrogenation reactor in the presence of hydrogen and a hydrogenation catalyst to produce a hydrogenated effluent stream; splitting the hydrogenated effluent into a bypass stream and a first exchanger inlet stream; varying a hydrogenated effluent throughput in the bypass stream; cooling the first exchanger inlet stream in a steam generator to produce a cooled hydrogenated effluent stream and a steam stream; adjusting a pressure of the steam stream produced in the steam generator to control the temperature of the cooled hydrogenated effluent stream. . An embodiment of the present disclosure is one, any or all of prior embodiments in this paragraph up through the second embodiment in this paragraph, further comprising minimizing the adjustment in pressure of the steam generator to about 50 psi. An embodiment of the present disclosure is one, any or all of prior embodiments in this paragraph up through the second embodiment in this paragraph, wherein at most 40% of the hydrogenated effluent is sent to the bypass stream. An embodiment of the present disclosure is one, any or all of prior embodiments in this paragraph up through the second embodiment in this paragraph, combining the bypass stream with the cooled hydrogenated effluent stream downstream of the steam generator into a combined feed stream and charging the combined feed stream to the second hydrogenation reactor. An embodiment of the present disclosure is one, any or all of prior embodiments in this paragraph up through the second embodiment in this paragraph, wherein the hydrogen is obtained from renewable energy sources.

A third embodiment of the present disclosure is a process for generating power in a hydrogenation process, the steps comprising, monitoring a dew point of a hydrocarbon feed stream at an inlet of a hydrogenation reactor; maintaining a dew point margin by adjusting a reactor inlet temperature of the hydrogenation reactor; hydrogenating the hydrocarbon feed stream in the hydrogenation reactor in the presence of hydrogen and a hydrogenation catalyst to produce a hydrogenated effluent; passing the hydrogenated effluent through a superheater exchanger to produce a superheated steam stream; and generating power in a turbine through the superheated steam stream.

An embodiment of the present disclosure is one, any or all of prior embodiments in this paragraph up through the second embodiment in this paragraph, wherein the dew point margin of the hydrocarbon feed stream is monitored using one or more dew point monitors. An embodiment of the present disclosure is one, any or all of prior embodiments in this paragraph up through the second embodiment in this paragraph, wherein the one or more dew point monitors are provided at the inlet of the hydrogenation reactor. An embodiment of the present disclosure is one, any or all of prior embodiments in this paragraph up through the second embodiment in this paragraph, wherein the dew point margin of the hydrocarbon feed stream is monitored to ensure maximum capture and superheating of the superheated steam stream in order to generate power in the turbine. An embodiment of the present disclosure is one, any or all of prior embodiments in this paragraph up through the second embodiment in this paragraph, wherein the reactor inlet temperature of the hydrogenation reactor is adjusted to maintain the dew point margin of the hydrocarbon feed stream based on a dew point margin set point above the reactor inlet temperature. An embodiment of the present disclosure is one, any or all of prior embodiments in this paragraph up through the second embodiment in this paragraph, wherein the dew point margin set point is about 20-25 °F. An embodiment of the present disclosure is one, any or all of prior embodiments in this paragraph up through the second embodiment in this paragraph, wherein the power is generated in a condensing type steam driven turbine.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the present disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

1. A process for controlling heat transfer in a hydrogenation process, the steps comprising:
hydrogenating a first hydrocarbon feed stream in a hydrogenation reactor in the presence of hydrogen and a hydrogenation catalyst to produce a hydrogenated effluent stream;
cooling the hydrogenated effluent stream in a steam generator to produce a cooled hydrogenated effluent stream and a steam stream; and
adjusting a pressure of the steam stream produced in the steam generator to control the temperature of the cooled hydrogenated effluent stream.

2. The process of claim 1 further comprising:
splitting the hydrogenated effluent stream into a bypass stream and a first exchanger inlet stream and cooling the first exchanger inlet stream in the steam generator; and
combining the bypass stream with the cooled hydrogenated effluent stream downstream of the steam generator into a combined feed stream and charging the combined feed stream to the second hydrogenation reactor.

3. The process of claim 2 wherein the temperature of the combined feed stream is also controlled by adjusting the flow rate of the bypass stream around the steam generator.

4. The process of claim 1, further comprising minimizing the adjustment in pressure of the steam generator to about 50 psi.

5. The process of claim 2, further comprising varying a hydrogenated effluent throughput in the bypass stream, wherein at most 40% of the hydrogenated effluent is sent to the bypass stream.

6. The process of claim 1, wherein hydrogen is obtained from renewable energy sources.

7. The process of claim 1, further comprising monitoring a dew point of the hydrocarbon feed stream at an inlet of the hydrogenation reactor.

8. The process of claim 7, further comprising maintaining a dew point margin by adjusting a reactor inlet temperature of the hydrogenation reactor.

9. The process of claim 1, further comprising:
superheating the steam stream in a heat exchanger with a second hydrogenated effluent stream to produce a superheated steam stream and a cooled second hydrogenated effluent stream; and
generating power in a turbine through the superheated steam stream.

10. The process of claim 9, wherein the heat exchanger is a superheater exchanger.

11. A process for controlling heat transfer in a hydrogenation process, the steps comprising:
hydrogenating a first hydrocarbon feed stream in a hydrogenation reactor in the presence of hydrogen and a hydrogenation catalyst to produce a hydrogenated effluent stream;
splitting the hydrogenated effluent into a bypass stream and a first exchanger inlet stream;
varying a hydrogenated effluent throughput in the bypass stream;
cooling the first exchanger inlet stream in a steam generator to produce a cooled hydrogenated effluent stream and a steam stream;
adjusting a pressure of the steam stream produced in the steam generator to control the temperature of the cooled hydrogenated effluent stream.

12. The process of claim 11, further comprising minimizing the adjustment in pressure of the steam generator to about 50 psi.

13. The process of claim 11, wherein at most 40% of the hydrogenated effluent is sent to the bypass stream.

14. The process of claim 11, combining the bypass stream with the cooled hydrogenated effluent stream downstream of the steam generator into a combined feed stream and charging the combined feed stream to the second hydrogenation reactor.

15. The process of claim 11, wherein the hydrogen is obtained from renewable energy sources.

16. A process for generating power in a hydrogenation process, the steps comprising:
monitoring a dew point of a hydrocarbon feed stream at an inlet of a hydrogenation reactor;
maintaining a dew point margin by adjusting a reactor inlet temperature of the hydrogenation reactor;
hydrogenating the hydrocarbon feed stream in the hydrogenation reactor in the presence of hydrogen and a hydrogenation catalyst to produce a hydrogenated effluent;
passing the hydrogenated effluent through a superheater exchanger to produce a superheated steam stream; and
generating power in a turbine through the superheated steam stream.

17. The process of claim 16, wherein the dew point of the hydrocarbon feed stream is monitored using a dew point monitor and wherein the dew point monitor is provided at the inlet of the hydrogenation reactor.

18. The process of claim 16, wherein the reactor inlet temperature of the hydrogenation reactor is adjusted to maintain the dew point margin of the hydrocarbon feed stream based on a dew point margin set point above the reactor inlet temperature.

19. The process of claim 18, wherein the dew point margin set point is about 20 to about 25 °F.

20. The process of claim 16, wherein the power is generated in a condensing type steam driven turbine.
